(19) United States
(12) Patent Application Publication
(10) Pub. No.: US 2002/0068090 A1
(43) Pub. Date: Jun. 6, 2002

(54) Calcium phosphate particles as mucosal adjuvants

(76) Inventors: Steve J. D. Bell, Marietta, GA (US); Qing He, Atlanta, GA (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 09/932,538

(22) Filed: Aug. 17, 2001

Related U.S. Application Data
(63) Continuation-in-part of application No. 09/496,771, filed on Feb. 3, 2000.
(60) Provisional application No. 60/118,356, filed on Feb. 3, 1999.
(60) Provisional application No. 60/118,364, filed on Feb. 3, 1999.
(60) Provisional application No. 60/118,355, filed on Feb. 3, 1999.

(51) Int. Cl. A61K 39/245; A61K 9/14

(52) U.S. Cl. 424/491; 424/231.1

(57) ABSTRACT

The present invention relates to mucosal immune protection, mucosal vaccine delivery, and mucosal drug delivery. Novel calcium phosphate core particles, particularly nanoparticles are used as vaccine adjuvants and compositions for inducing protective mucosal immunity. Methods of inducing an immune response to an antigen by delivering the antigen to a mucosal surface using the particles of this invention, and to methods of making such particles are also provided. The particles of this invention are also useful for delivering compositions, such as a pharmacologically active agent, to the mucosal surfaces of a patient in need thereof, to methods of delivering such compositions, and to methods of making such particles.

This application is a continuation-in-part of U.S. patent application Ser. No. 09/496,771 filed on Feb. 3, 2000, which claims benefit of the filing dates of U.S. Provisional Application Ser. Nos. 60/118,356; 60/118,364; and 60/118,355, all filed Feb. 3, 1999, the entire contents of each of which are hereby incorporated by reference.

### BACKGROUND OF INVENTION

1. Field of the Invention

The present invention relates to mucosal immune protection, mucosal vaccine delivery, and mucosal drug delivery. Particularly, the invention relates to novel calcium phosphate core particles, particularly nanoparticles, as vaccine adjuvants and compositions for inducing protective mucosal immunity. The invention also relates to methods of inducing an immune response to an antigen by delivering the antigen to a mucosal surface using the particles of this invention, and to methods of making such particles. The novel calcium phosphate core particles of this invention are also useful for delivering compositions, such as a pharmacologically active agent, to the mucosal and secretory surfaces of a patient in need thereof, to methods of delivering such compositions, and to methods of making such particles.

2. Description of Related Art

The human immune system may be broadly subdivided into two separate, interacting subsystems. The central immune system patrols the inner organs and tissues, and the mucosal immune system provides a defensive barrier against microbes that enter the body through the surfaces of the airways, intestines and urogenital tract. While the central immune system has been the subject of intensive study over the last three decades, knowledge of the mucosal immune system remains poor.

Additionally, since mucosal surfaces act as the primary route of invasion for most pathogens and the first line of defense against them, vaccines inducing effective mucosal immunity may reduce rates of infection, and decrease the morbidity and mortality of infectious diseases. Currently, no other safe and effective mucosal adjuvant has received regulatory approval for human use.

Many pathogenic bacteria and viruses initially gain entry into the body by crossing the cellular linings (epithelia) of the gastrointestinal, respiratory, or genital tracts. A specialized class of antibodies, IgA antibodies, protects these surfaces. IgA antibodies are dimeric or dimeric molecules produced by cells located in the tissues under the epithelial surfaces. They are transported by epithelial cells into mucosal secretions, where they crosslink or coat pathogens that have not yet entered the body, thus preventing the pathogens from contacting and adhering to epithelial cells. Thus, IgA antibodies operate on pathogens that are outside the body, and they protect by preventing entry into the body across epithelial surfaces.

The naturally occurring IgA response is triggered by antigen contacting the mucosal surfaces. The antigen enters the body through specific sampling sites (called microfold or M cells) that effect transepithelial antigen transport to areas of the mucosal lining containing specialized, organized collections of the cells of the mucosal immune system. More specifically, antigens bind the luminal surface of M cells and are internalized and transcytosed to be released in the intra-epithelial pocket containing lymphoid cells (B and T cells) and antigen-processing/presenting cells, such as macrophage cells.

IgA antibodies in a naturally immunized host are transported into secretions by binding to a specific receptor (called the poly-Ig receptor) on the basal (interior) surfaces of epithelial and glandular cells throughout the respiratory and digestive systems, the genital tract, and the mammary glands. Receptor-IgA complexes are transported across these cells and exocytosed onto luminal (exterior) cell surfaces where the receptor is enzymatically cleaved, releasing IgA into secretions along with a receptor fragment called secretory component (SC).

In general, existing immunization strategies which involve injection of antigens evoke production of the IgG class of antibodies that circulate systemically and neutralize pathogens after they have entered the body. Injection of antigens does not generally evoke a substantial IgA response. Efforts to take advantage of IgA protection at mucosal barriers have included oral immunization, as well as applying monoclonal IgA antibodies directly to respiratory mucosal surfaces in an effort to protect against pathogen entry. Active immunization may involve active or passive encounter at the mucosal surface with intact (killed) bacteria or viruses. To avoid dangers that may be associated with exposure of the host to live or live attenuated pathogens preparations for immunization, component antigens, such as immunogenic native or recombinant analogs of surface components of the pathogen, may be applied at a mucosal surface.

Some antigens are highly immunogenic and as stand-alone entities, are capable alone of eliciting protective immune responses. Other antigens, however, fail to induce a protective immune response or induce only a weak immune response. Frequently, the host immune response to a weakly immunogenic antigen can be significantly enhanced if the antigens are co-administered with adjuvants. Adjuvants enhance the immunogenicity of antigens but are not necessarily immunogenic themselves. Adjuvants may act by retaining the antigen locally near the site of administration to produce a depot effect facilitating a slow, sustained release of antigen to cells of the immune system. Adjuvants can also attract cells of the immune system to an antigen depot and stimulate such cells to elicit immune responses. Adjuvants have been identified that enhance the immune response to antigens delivered parenterally, but there is little research addressing adjuvants to enhance mucosal immune responses.

Accordingly, there is a need in the art to provide effective mucosal adjuvants, as well as effective mucosal vaccine delivery. Studies have established that immune stimulation of mucosa in one part of the body can elicit a similar antibody response in mucosal regions remote from the initial antigen exposure, a concept termed the common mucosal immune system. See McGhee JR, et al., The mucosal immune system: from fundamental concepts to vaccine development, Vaccine 1992; 10:75-88. For example, intranasal immunization of a variety of vaccine compositions has been reported to favor the development of B cell responses at distal mucosal sites, for example, resulting in vaginal antibody secretion. Among such vaccines are a recombinant Salmonella typhimurium vaccine expressing a hepatitis virus antigen, a streptococcal surface antigen coupled to the cholera toxin (CT) B subunit, an HIV peptide given with CT, a replication competent adenoviral recombinant expressing a herpes virus antigen, and a DNA vaccine with CT as an adjuvant. See U.S. Pat No. 6,210,633.

However, the limited understanding of the mucosal immune system has hampered the development of efficacious vaccines to numerous important human pathogens which infect the host via mucosal surfaces, such as the genital tract mucosal surface, the nasal mucosal surface, and so on. Despite the above reports, vaccines which induce effective mucosal immunity, particularly vaccines to induce protective immunity against sexually transmitted pathogens, which infect humans via the mucosa and which constitute major emerging and re-emerging infectious diseases, are not currently available. For example, agents causing emerging and re-emerging infectious diseases include human immunodeficiency virus (HIV), human papilloma viruses (HPV), Neisseria gonorrhea, Treponema pallidum, and herpes simplex virus types 1 and 2 (HSV-1 and HSV-2). These agents often establish persistent infections once they have avoided the mucosal barrier.

Vaccines inducing mucosal immunity are essential to preventing the transmission of infection via mucosal surfaces, the primary route of entry into the body. However, no mucosal vaccine adjuvant is currently approved for human use. Because the immunogenicity of some antigens targeted for vaccine development, such as recombinant subunitepitopes, and native or recombinant peptides, are generally relatively weak, finding efficient vaccine adjuvants represents a highly desirable strategy to achieve protective mucosal immunity. The only adjuvants used in licensed vaccines in the United States are aluminum (alum) compounds. Although alum adjuvants and their derivatives sometimes enhance immune responses to protein subunits effectively, frequently, as human studies have shown, the cell mediated immunity and antibody responses they elicit tend to be weak, inadequate for protection, or may even be non-existent. Additionally, alum has a marked propensity to elicit aberrant IgE type antibody responses to vaccine antigens, which are associated with allergic reactions.

Thus, a major obstacle to developing a mucosal vaccine in humans is finding an effective and safe adjuvant. Current experimental mucosal adjuvants include cholera toxin (CT), heat-labile enterotoxin (LT), mutant toxin (LTK63 and LTR72), cytosine-guanine dinucleotides (CpG), polymerized liposomes, microparticles, biodegradable microspheres and recombinant interleukins or immune modulators(IL). Each of these adjuvants is still in early development, and none has been approved for use in humans. Singh M, O'Hagan DT. Advances in vaccine adjuvants. Nature Biotech 1999; 17:1075-1081.

Biodegradable and biocompatible calcium phosphate particles have been investigated as an alternative to aluminum adjuvants in parenteral vaccines and have been used in France to enhance secondary or booster immunizations against diphtheria and tetanus in humans. See Ickovic MR, Relyveld EH, Henocq E, Calcium phosphate adjuvanted allergens: Total and specific IgE levels before and after immunotherapy with house dust and mite extracts, Ann. Immunolo. (Inst. Pasteur) 1983; 134(D):385-98; Neefjes JJ, Mornburg F, Cell biology of antigen presentation, Curr. Opin. Immunolo. 1993; 5(1):27-34. Calcium phosphate has also been used for allergen desensitization. See Powell MF, Newman M. J. Adjuvant properties of aluminum and calcium compounds, Vaccine Design 1995: 229-48; Relyveld, EH. Preparation and use of calcium phosphate adsorbed vaccines, Develop. Biol. Standard 1986; 65:131-136. See id.; Relyveld EH, Ickovic MR, Henocq E, Garcelon M. Calcium phosphate adjuvanted allergens. Ann Allergy 1985; 54(6):11-19. Although these earlier studies using calcium phosphate suggested that CAP had immune modulating capabilities (for example, when used to decrease Th2-T-cell-associated IgE allergic responses), CAP's use as a selective Th1-T-cell-associated enhance of microbial immunity, especially mucosal IgA production and anti-viral cell-mediated immunity (CMI), was unknown.

The present inventors' early studies with nanoparticulate calcium phosphate indicated that these particles produce strong Th1 T-cell-associated and mucosal IgA immunity. In strong contrast to aluminum adjuvants, which generally trigger production of IgE antibody and produce local irritation at the site of injection in animal experiments and human clinical trials, CAP does not elicit significant IgE responses, and safety and toxicity studies indicate that CAP does not trigger significant irritation at the site of injection.

One example of the use of hydroxylated calcium phosphate (hydroxyapatite) for administering an immunogen to a mucosal surface of a mammal, is described by U.S. Pat. No. 5,443,832, incorporated herein by this reference. Commercially available particles comprised of hydroxyapatite, a modified form of crystalline calcium phosphate, were broken into small crystalline fragments less than 1 μm, preferably between about 0.01 and 0.1 μm. The amorphic hydroxyapatite crystals were directly coated with proteins, without description of any additional surface modifying agents or binding materials and without suggestion of entrapping the proteins within the particles, as disclosed by the present invention. Moreover, these modified hydroxyapatite crystals have a very different chemical composition from the particles of the present invention. By virtue of the amorphous physical character and the different in vivo dissolution properties that are likely to occur because of differing chemical composition (and thus density differences), the nature of the immune responses is different. For example, the present inventors' results from comparative animal studies, in which the same modified hydroxyapatite was used to conduct comparative studies against the CAP particles of the present invention showed that the modified hydroxyapatite consistently elicited stronger Th2-T-cell associated immune responses and elicited IgE production.

Nanometer scale particles have been proposed for use as carrier particles, as supports for biologically active molecules, such as proteins, and as decoy viruses. See U.S. Pat. Nos. 5,178,882; 5,219,577; 5,306,508; 5,334,394; 5,460,830; 5,460,831; 5,462,750; and 5,464,634, the entire contents of each of which are hereby incorporated by reference.

The particles disclosed in the above-referenced patents, however, are generally extremely small, in the 10-200 nm size range. Particles of this size can be difficult to make with consistency, and their morphology is not described in any detail. These patents do not disclose the use of nanoparticles as controlled release matrices Furthermore, these patents do not disclose the use of calcium phosphate particles as either (1) adjuvants for vaccines or viral decoys, or (2) controlled release matrices for delivery of bioactive pharmaceuticals or immunogenic materials.

Scientific reports have suggested a use for calcium phosphate particles as vaccine adjuvants, but those calcium phosphate particles have generally been considered an unsuitable alternative to other adjuvants due to inferior adjuvanting activity. See, e.g., Goto et al., Vaccine, vol. 15, no. 12/13 (1997). One of the more important distinctions between the previously-studied calcium phosphate particles and those of the present invention is that the chemical compositions and physical characteristics of the former calcium phosphate particles is markedly different from the particles of the present invention—hence, the former's inferior adjuvanting activity. Moreover, the calcium phosphate evaluated in that reference was typically microparticulate (>1000 nm diameter) and possessed a rough and oblong morphology, in contrast to the core particles of the present invention.

PCT Application No. WO 00/15194 to Lee et al. published on Mar. 23, 2000 also discusses calcium phosphate particles for delivery vehicles and adjuvants. This reference does not provide an adequate description of the use of its particle as a mucosal adjuvant, vaccine, or drug delivery agent. Moreover, the particles of the this reference would be difficult to manufacture. That is, the method involves multiple steps and time-consuming, labor-intensive, and costly intervening procedures.

Therefore, an important need remains for calcium phosphate core particles useful as core materials or carriers which can be produced simply and consistently for biologically active moieties. A further need remains for calcium phosphate core particles that can be effectively used as mucosal adjuvants for vaccines, as cores or carriers for biologically active molecules, and as controlled release matrices, as a vaccine delivery vehicles for delivering antigens to the mucosal surfaces, and thusto generate mucosal immunity, and as delivery vehicles for delivering pharmacologically active agents to mucosal surfaces.

There is also a need for calcium phosphate core particles that can be effectively used as supports and matrices for sustained release of polynucleotide material (DNA or RNA) encoding immunogenic polypeptides. Traditional vaccination involves exposing a potential host to attenuated or killed pathogens, or immunogenic components thereof (e.g., proteins or glycoproteins). The basic strategy has changed little since the development of the first smallpox vaccine nearly a century ago, although modem developments permit genetic engineering of recombinant protein vaccines. However, traditional vaccine methodologies may be undesirable as a result of their expense, instability, poor immunogenicity, limited heterogeneity and in the case of live-attenuated vaccines, might render vaccine recipients susceptible to pathogenic infection.

Vaccination with oligonucleotides presents a different vaccine methodology, whereby oligonucleotide material, such as DNA or RNA gene sequences, encoding an immunogenic polypeptide is delivered intracellularly to a potential host. The genetic material is taken up, translated intracytoplasmically, and the antigenic peptide products expressed by these cells—this approach has the potential to result in both a humoral and a cell-mediated immune responses. It is not entirely clear whether DNA vaccines function as a result of integration or simply long-term episomal maintenance.

Oligonucleotide vaccination provides numerous advantages over traditional vaccination. Oligonucleotide vaccines eliminate the risk of infection associated with vaccination with liveattenuated viruses, yet advantageously induce both humoral and cell-mediated responses. Oligonucleotide vaccines further provide prolonged immunogen expression, generating significant immunological memory and eliminating the need for multiple inoculations. Oligonucleotide vaccines are very stable, permitting prolonged storage, transport and distribution under variable conditions. As a further advantage, a single oligonucleotide vaccine may be engineered to contain gene sequences that code for multiple immunogenic polypeptide products. Thus, a single DNA vaccine can be used to immunize against multiple pathogens, or multiple strains of the same pathogen. Finally, oligonucleotide vaccines are much simpler and less expensive to manufacture than traditional vaccines.

Oligonucleotide vaccines may take various forms. The genetic material can be provided, for example, in combination with adjuvants capable of stimulating the immune response. Administration of the DNA or RNA coated onto microscopic beads has been suggested. See J. J. Donnelly et al., Annu. Rev. Immunol. 15, 617 (1997). Various routes of administration are also possible, and may include, for example, intravenous, subcutaneous and intramuscular administration. It has not been suggested, however, to deliver DNA or RNA encoding immunogenic polypeptides through mucosal routes. One advantage of mucosal administration is that it is non-invasive and thus less painful to the vaccinee, relative to the classic parenteral modes of vaccination.

A desirable immune response to an immunogenic polypeptide is two-fold, involving both humoral and cell-mediated immunity, with the mucosal immunity manifested as a humoral response. The humoral component is manifested following stimulation of B cells which differentiate into antibody-producing plasma cells, and antibodies are capable of recognizing extracellular pathogens, while the cell-mediated component involves T lymphocytes capable of recognizing intracellular pathogens. Cytotoxic T-lymphocytes (CTLs) play an important role in the latter, by lysing virally-infected or bacterially-infected cells. Specifically, CTLs possess receptors capable of recognizing foreign peptides associated with MHC class I and/or class II molecules. These peptides, in the case of recombinant DNA or RNA vaccines, are typically the translation products of these vaccine candidates. In contrast, in the case of natural infections, these peptides can be derived from endogenously synthesized foreign proteins, regardless of the protein's location or function within the pathogen. Thus, CTLs can recognize epitopes derived from conserved internal viral proteins (J. W. Yewdell et al., Proc. Natl. Acad. Sci. (USA) 82, 1785 (1985); A. R. M. Townsend, et al., Cell 44, 959 (1986); A. J., McMichael et al., J. Gen. Virol. 67, 719 (1986); A. R. M. Townsend and H., Annu. Rev. Immunol. 7, 601 (1989)) and may therefore permit heterologous protection against viruses with multiple serotypes or high mutation rates. Oligonucleotide vaccination can stimulate both forms of immune response, and thus is very desirable.

Efforts to use oligonucleotide vaccination have focused on the use of viral vectors to deliver oligonucleotides to host cells. J. R. Bennink et al., 311, 578 (1984); J. R. Bennink and J. W. Yewdell, Curr. Top. Microbiol. Immunol. 163, 153 (1990); C. K. Stover et al., Nature 351, 456 (1991); A. Aldovini and R. A. Young, Nature 351, 479 (1991); R. Schafer et al., J. Immunol. 149, 53 (1992); C. S. Hahn et al., Proc. Natl. Acad. Sci. (USA) 89, 2679 (1992). However, this approach may be undesirable for several reasons. Retroviral vectors, for example, have restrictions on the size and structure of polypeptides that can be expressed as fusion proteins while maintaining the ability of the recombinant virus to replicate (A. D. Miller, Curr. Top. Microbiol. Immunol. 158, 1 (1992). The effectiveness of vectors such as vaccinia for subsequent immunizations may be compromised by immune responses against vaccinia (E. L. Cooney et al., Lancet 337, 567 (1991)). Also, viral vectors and modified pathogens have inherent risks that may hinder their use in humans (R. R. Redfield et al., New Engl. J. Med. 316, 673 (1987); L. Mascola et al., Arch. Intern. Med. 149, 1569 (1989)). For example, in live vector approaches, highly immunogenic vectors also tend to be highly pathogenic, particularly when administered to immunocompromised hosts.

Alternative gene delivery methods have also been explored. Benvenisty, N., and Reshef, L. (PNAS 83, 9551-9555, (1986)) showed that CaCl₂ precipitated DNA could be expressed in mice. Plasmid vectors have also been used to produce expression in mouse muscle cells (J. A. Wolff et al., Science 247, 1465 (1990); G. Ascadi et al., Nature 352, 815 (1991)). The plasmids were shown to be maintained episomally and did not replicate. Subsequently, persistent expression has been observed after i.m. injection in skeletal muscle of rats, fish and primates, and cardiac muscle of rats (H. Lin et al., Circulation 82, 2217 (1990); R. N. Kitsis et al., Proc. Natl. Acad. Sci. (USA) 88, 4138 (1991); E. Hansen et al., FEBS Lett. 290, 73 (1991); S. Jiao et al., Hum. Gene Therapy 3, 21 (1992); J. A. Wolff et al., Human Mol. Genet. 1, 363 (1992)). WO 90/11092 (Oct. 4, 1990) reported the use of naked polynucleotides to vaccinate vertebrates.

Various routes of administration have been found to be suitable for vaccination using oligonucleotide vaccines. Intramuscular administration is thought to be particularly desirable, given the proportionally large muscle mass and its direct accessibility through the skin. See U.S. Pat. No. 5,580,859. Tang et al., (Nature, 356, 152-154 (1992)) disclosed that introduction of gold microprojectiles coated with DNA encoding bovine growth hormone (BGH) into the skin of mice resulted in production of anti-BGH antibodies in the mice. Furth et al., (Analytical Biochemistry, 205, 365-368, (1992)) showed that a jet injector could be used to transfect skin, muscle, fat, and mammary tissues of living animals. WO 93/17706 describes a vaccination method wherein carrier particles are coated with a gene construct and then accelerated into a potential host. Intravenous injection of a DNA:cationic liposome complex in mice has also been reported (Zhu et al., Science 261, 209-211 (Jul. 9, 1993); see also WO 93/24640). Methods for introducing nucleic acids have been reviewed (Friedman, T., Science, 244, 1275-1281 (1989)); see also Robinson et al., (Abstracts of Papers Presented at the 1992 meeting on Modem Approaches to New Vaccines, Including Prevention of AIDS, Cold Spring Harbor, p 92; Vaccine 11, 957 (1993)), where the intra-muscular, intra-venous, and intra-peritoneal administration of avian influenza DNA into chickens was alleged to have provided protection against lethal challenge.

Reports suggest that oligonucleotide vaccination has provided effective protective immunity in various animal models. The immunization of mice against influenza by the injection of plasmids encoding influenza A hemagglutinin has been reported (Montgomery, D. L. et al., 1993, Cell Biol., 12, pp. 777-783), or nucleoprotein (Montgomery, D. L. et al., supra; Ulmer, J. B. et al., 1993, Science, 259, pp. 1745-1749). The first use of DNA immunization for a herpes virus has been reported (Cox et al., 1993, J. Virol., 67, pp. 5664-5667). Injection of a plasmid encoding bovine herpes virus 1 (BHV-1) glycoprotein g IV gave rise to anti-g IV antibodies in mice and calves. Upon intranasal challenge with BHV-1, immunized calves showed reduced symptoms and shed substantially less virus than controls.

Wang et al., (P.N.A.S. USA 90, 4156-4160 (May, 1993)) reported on elicitation of immune responses in mice against HIV by intramuscular inoculation with a cloned, genomic (unspliced) HIV gene. However, the level of immune responses achieved was very low, and the system utilized portions of the mouse mammary tumor virus (MMTV) long terminal repeat (LTR) promoter and portions of the simian virus 40 (SV40) promoter and terminator. SV40 is known to transform cells (a major safety concern a form of cancer), possibly through integration into host cellular DNA. Thus, the system described by Wang et al., may be inappropriate for administration to humans.

It has been suggested to use calcium phosphate particles as agents for transfection of therapeutic polynucleotides in gene therapy. See U.S. Pat. No. 5,460,831. DNA or RNA is attached to the particulate core and delivered to a target cell, resulting in expression of therapeutic proteins. However, this patent does not suggest the use of calcium phosphate particles as supports for DNA or RNA vaccines. To the contrary, this patent indicates that the stimulation of an immunological response during transfection is to be avoided. This patent also fails to suggest the use of calcium phosphate particles as controlled release matrices for genetic material, or for the mucosal delivery of such controlled release matrices.

There is also a need for calcium phosphate core particles that can be applied to mucosal surfaces, such as intraocularly, intravaginally, intranasally, and so on, as a vaccine adjuvant to generate mucosal immunity. For a number of therapeutic agents, delivery of the agent to a patient in need thereof can be difficult. This is particularly true with proteins and peptides, which are difficult or impossible to administer orally, since they are easily digested or hydrolyzed by the enzymes and other components of gastric juices and other fluids secreted by the digestive tract. Injection is often the primary alternative administration method, but is unpleasant, expensive and is not well tolerated by patients requiring treatment for chronic illnesses. In particular, patients who are administered drugs on an out-patient basis, or who self-administer, are more likely to fail to comply with the required administration schedule.

Recently, alternative methods of administration of therapeutic agents and alternate routes for developing effective mucosal immunity have been sought. In particular, some studies have focused on administration of therapeutic agents by inhalation of an aerosol containing a therapeutic agent. The lungs can be used effectively to deliver the therapeutic agent to the bloodstream because they have a very large surface area of very thin tissue. As a result, for some therapeutic agents and delivery systems, the level of agent in the blood can rise as fast as, or faster than, that obtained when the agent is administered by injection beneath the skin. Moreover, the thin lung tissue allows the passage of proteins and peptides into the blood stream without exposing them to the type or level of proteases encountered during oral administration.

Additionally, researchers have studied methods and compositions for delivering drugs, such as peptides, and antigenic vaccine ingredients, for delivery across a mucosal surface such as the vagina, eye or nose. See U.S. Pat. 5,204,108. This reference describes microspheres between 10 and 100 microns that gel in contact with a mucosal surface. The microspheres have the active agent incorporated into the microsphere or sorbed onto/into the microsphere. The microsphere delivery systems may also include microspheres made from the active peptide or protein itself, such as insulin microspheres. Non-ionic surfactant vesicles have also been used to potentiate an immunological response to antigens. See U.S. Pat. No. 5,679,355. Other studies have focused on immunizing hosts against infection by delivering vaccine compositions for intranasal administration of an influenza vaccine. See U.S. Pat. No. 6,048,536.

Recent comparative studies have indicated that microparticles are potent adjuvants for mucosal delivery. However, microparticles are not in an ideal size range for inducing cellular immunity since they traditionally have been too large, and it is believed that dendritic cells, macrophages and local lymph nodes, following inoculation, can more easily take up smaller particles. Other studies have shown that microparticles may preferentially induce cytotoxic T cell lymphocyte (CTL) proliferation compared to alum. The CAP nanoparticles of the present invention are smaller than about 1200 nanometers in size, and also appear to stimulate CTL immunity.

Despite the above-described attempts, there remains a long-felt and acute need for the design and development of efficacious vaccines to induce or confer mucosal immunity in humans to human pathogens. The present inventors have developed a CAP vaccine adjuvant that induces both systemic and mucosal immunity, and given its relative absence of side effects and lack of IgE antibody induction, that is safer and potentially more useful as a mucosal vaccine adjuvant. The CAP particles of the present invention may also be useful for delivering any pharmacologically active agent to mucosal surfaces.

### SUMMARY OF THE INVENTION

The present invention relates to a unique formulation of calcium phosphate nanoparticles (CAP) for use as a mucosal adjuvant. The particles are particularly useful as carriers for native and recombinant antigens or any other pharmacologically active native or recombinant agent to be applied to mucosal surfaces. The present inventors have found that immunization with CAP-based formulations of an antigen, for example herpes simplex-2 (HSV-2) glycoprotein or influenza M1 protein, exhibited significantly increased survival rates and less severe clinical infection compared to controls, following live-viral challenge. These findings demonstrate that CAP delivered as a mucosal adjuvant confers protective antiviral immunity.

More particularly, the invention relates to the core CAP particles having a diameter between about 300 nm and about 4000 nm, more particularly between about 300 nm and about 1000 nm, and having a substantially spherical shape and a substantially smooth surface, that can deliver a native or recombinant immunogen or other native or recombinant pharmacologically active agent to mucosal surfaces.

The present invention also relates to particles having a pharmacologically active agent coated on the surface of the core particles, and/or dispersed or impregnated within the core particles, to methods of making them, and to methods of using them. Non-limiting examples of a suitable pharmacologically active agent to be at least partially coated on the surface of the core particle or impregnated therein include one or more of the following: antigenic material, natural immunoenhancing factors, polynucleotide material encoding immunogenic polypeptides, therapeutic drugs, such as insulin, or any other composition capable of having a therapeutic effect when administered to a mucosal surface.

The present invention also relates to combinations of this novel core particle and pharmacologically active agent having at least a partial coating of a surface modifying agent or a surface modifying agent impregnated therein or both. If one or more of the above-mentioned pharmacologically active agents is at least partially coated on the particle, the agent may be optionally attached to the particle by the surface modifying agent, which acts as a biological ‘glue,’ such as cellobiose or polyethylene glycol (PEG).

More particularly, the present inventors have discovered a specific formulation of (CAP) calcium phosphate particles that are particularly useful as carriers for antigens to be applied to mucosal surfaces. The CAP combined with an antigen is transported across epithelium where it raises a protective antigen-specific mucosal antibody, and systemic humoral and cell-mediated immune responses.

The exact mechanism of the adjuvant action of CAP is not fully understood. M cells in the mucosal surface are known to reside exclusively in the epithelium and deliver foreign material by transepithelial transport from lumen to the underlying mucosa-associated lymphoid tissue. Particulate antigens are desirable since they permit M cells to translocate across the tight epithelial barrier to mucosal dendritic cells. Therefore, the particulate mucosal vaccine created from the combination of soluble antigens formulated within CAP provides the desirable size and functional attributes to induce effective mucosal immunity.

One aspect of the invention generally features a method for preparing the CAP particles combined with an antigen for mucosal delivery. The resulting particles may be used to elicit antigen-specific immunity in a mammal by delivering an immunogen comprising an antigen or an antigen mixture in association with the CAP particles, administered to a mucosal surface of the mammal.

A second aspect of the invention features a method for vaccinating a mammal (especially a human) comprising administering the above-described immunogen to a mucosal surface of the mammal. A third aspect of the invention features delivering any pharmacologically active agent in association with CAP particles of a size suitable for transport across epithelium.

The preferred modes of administration of the immunogen or other pharmacologically active agent according to this invention are through mucosal surfaces, such as orally, intrapulmonary, vaginally, nasally, rectally, or ocularly. The CAP formulations may be compounded with any physiologically acceptable vehicle or other complementary immunological adjuvant and applied directly to the mucosal surface tissue, e.g., to oral, pulmonary, nasal, ocular, rectal and/or vaginal surfaces.

The immunogen or other pharmacologically active agent can adhere to the mucosa and be transported efficiently across the epithelial barrier for presentation to the mucosal immune system, thus also having the potential to concurrently elicit an associated systemic or distal immune response. The proteins may be admixed with, or adsorbed to the CAP, complexed with the CAP such that the immunogen is entrapped within the particle, or both, to provide a sustained release model. Moreover, CAP has a high general affinity for the antigens of interest, including oligonucleotides, proteins and other antigens, making the invention broadly applicable.

CAP is non-toxic, non-immunogenic, and is easily degraded by the body, and accordingly, CAP can be safely administered, and administration can be repeated using the same CAP vehicle for the same or different antigens, without an anti-vehicle immune response. Moreover, the CAP particles of the present invention can be prepared relatively rapidly and inexpensively.

The present invention also relates to methods of treating medical conditions resulting from protein or peptide deficiencies by administering effective amounts of the core particles of this particular embodiment to a patient in need thereof via mucosal surfaces, such as intranasally, intrapulmonary, intraocularly, intravaginally, rectally, etc. The therapeutic compositions of the present invention are highly stable, and exhibit enhanced bioavailability.

The present invention also relates to methods of preparing the novel calcium phosphate core particles having an antigen or other pharmacologically active agent at least partially coated on the surface (“outside formulation”), impregnated therein (“inside formulation”), or both (“inside/outside formulation”).

The above discussed and many other features and attendant advantages of the present invention are detailed below. Other features and advantages of the invention will be apparent from the following description of the preferred embodiments thereof.

### BRIEF DESCRIPTION OF THE FIGS.

FIG. 1 is a schematic drawing showing a calcium phosphate core particle (4) both coated with an antigenic material or other pharmacologically active agent (8) and having antigenic material or other pharmacologically active agent (8) impregnated therein.

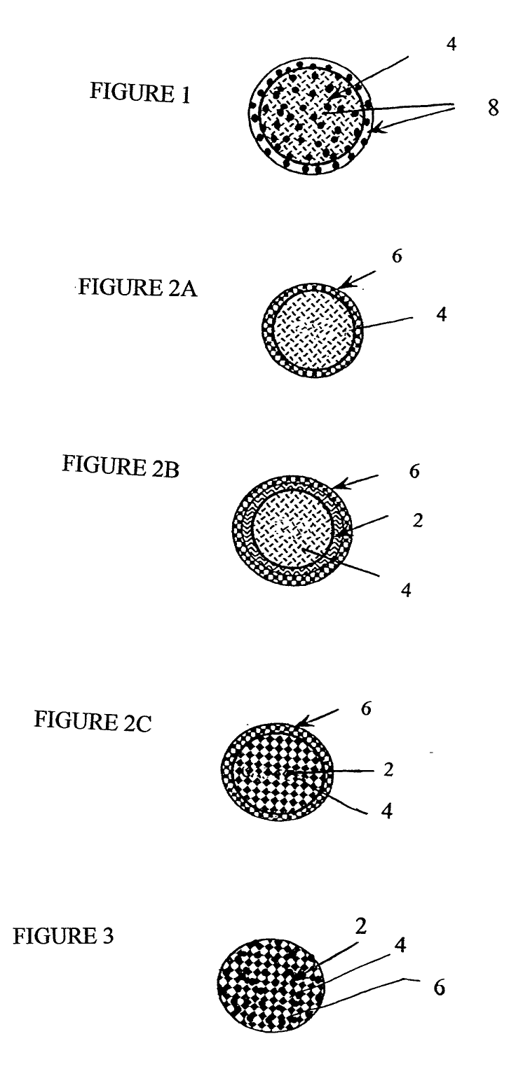

FIG. 2 is a series of schematic drawings showing various embodiments of calcium phosphate core particles. FIG. 2A shows a core particle coated directly with pharmacologically active agent (6). FIG. 2B shows a core particle (4) coated with surface modifying agent (2), such as polyethylene glycol or cellobiose, and a having a pharmacologically active agent (6) adhered to the surface modifying agent (2). FIG. 2C shows a calcium phosphate core particle (4) having a surface modifying agent (2), such as polyethylene glycol or cellobiose incorporated therein and having a pharmacologically active agent (6) at least partially coating core particle (4).

FIG. 3 is a schematic drawing showing a calcium phosphate core particle (4) having both a surface modifying agent (2), such as polyethylene glycol or cellobiose and a material (6), such as antigenic material, natural immunoenhancing factors, polynucleotide material encoding immunogenic polypeptides, or therapeutic proteins or peptides incorporated therein.

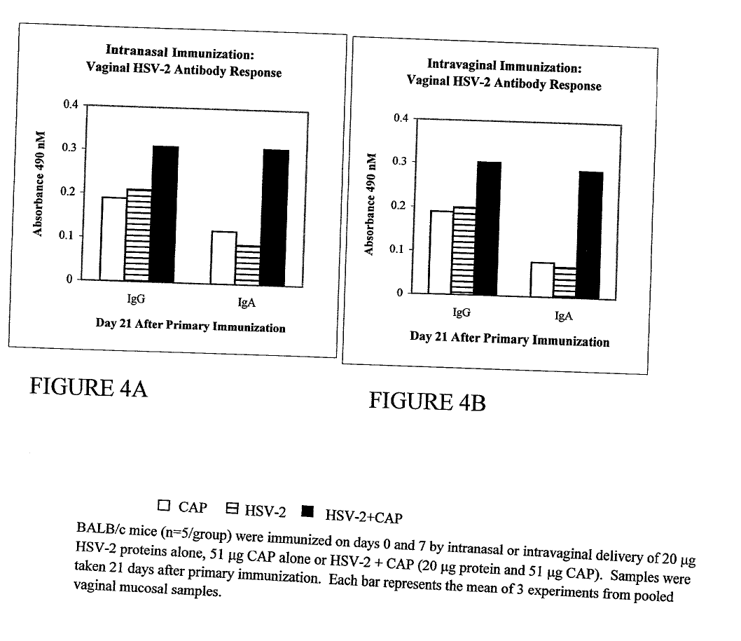

FIG. 4a shows the results from three vaccine formulations, HSV-2 alone, CAP alone, and HSV-2+CAP, administered intranasally with vaginal HSV-2 antibody response evaluated for antibody and protective responses in mice.

FIG. 4b shows the results from three vaccine formulations, HSV-2 alone, CAP alone, and HSV-2+CAP, administered intravaginally with vaginal HSV-2 antibody response evaluated for antibody and protective responses in mice.

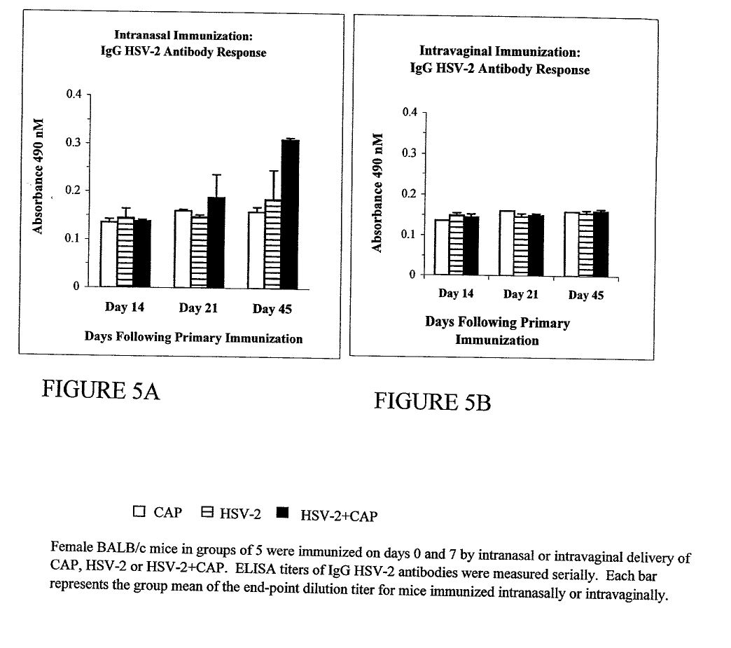

FIG. 5a shows the results from three vaccine formulations, HSV-2 alone, CAP alone, and HSV-2+CAP, administered intranasally with IgG HSV-2 antibody response evaluated for antibody and protective responses in mice.

FIG. 5b shows the results from three vaccine formulations, HSV-2 alone, CAP alone, and HSV-2+CAP, administered intravaginally with IgG HSV-2 antibody response evaluated for antibody and protective responses in mice.

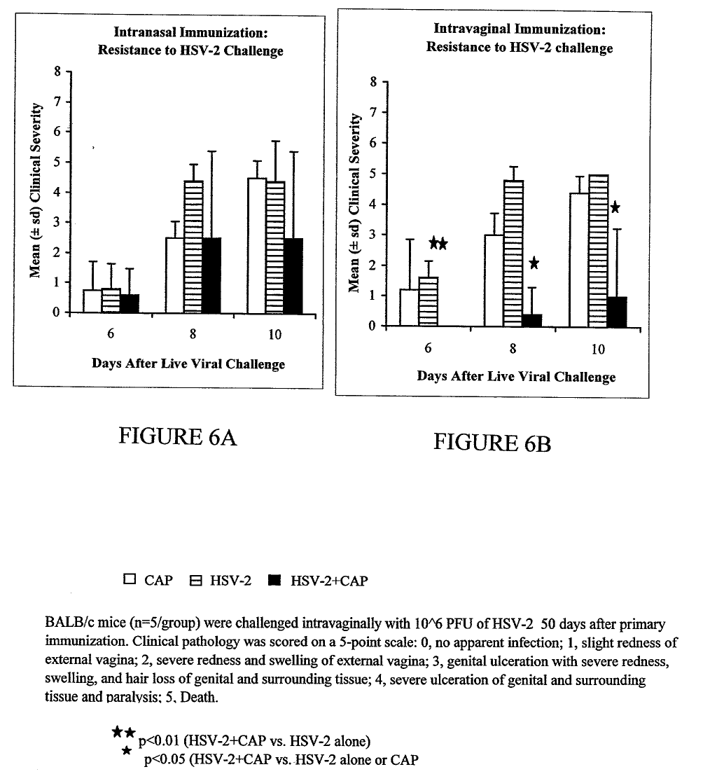

FIG. 6a shows the clinical pathology results from intravaginal challenge after intranasal vaccination with HSV-2+CAP as compared to HSV-2 or CAP alone.

FIG. 6b shows the clinical pathology results from intravaginal challenge with live virus, following intravaginal vaccination with HSV-2+CAP as compared to HSV-2 or CAP alone.

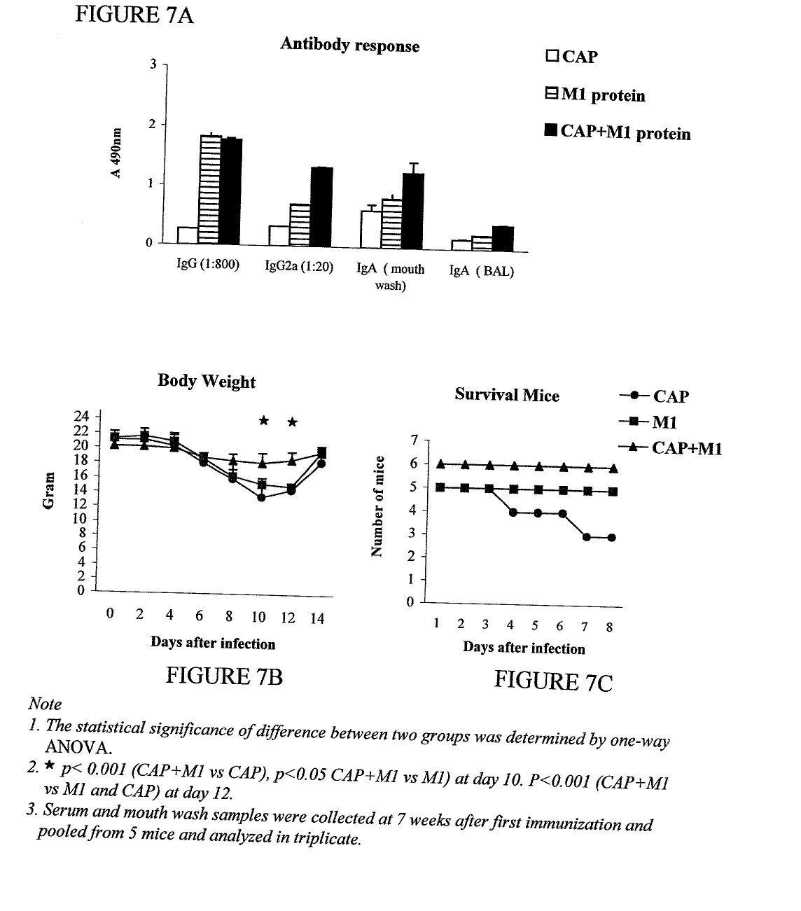

FIG. 7a shows the results from three vaccine formulations, M1 alone, CAP alone, and M1+CAP, with M1 antibody response evaluated for antibody and protective responses in mice.

FIG. 7b shows the results from the vaccine formulations administered in FIG. 7a, with morbidity evaluated by measuring body weight every other day.

FIG. 7c shows the results from the vaccine formulations administered in FIG. 7a, with the resulting survival rate for each vaccine formulation after challenge.

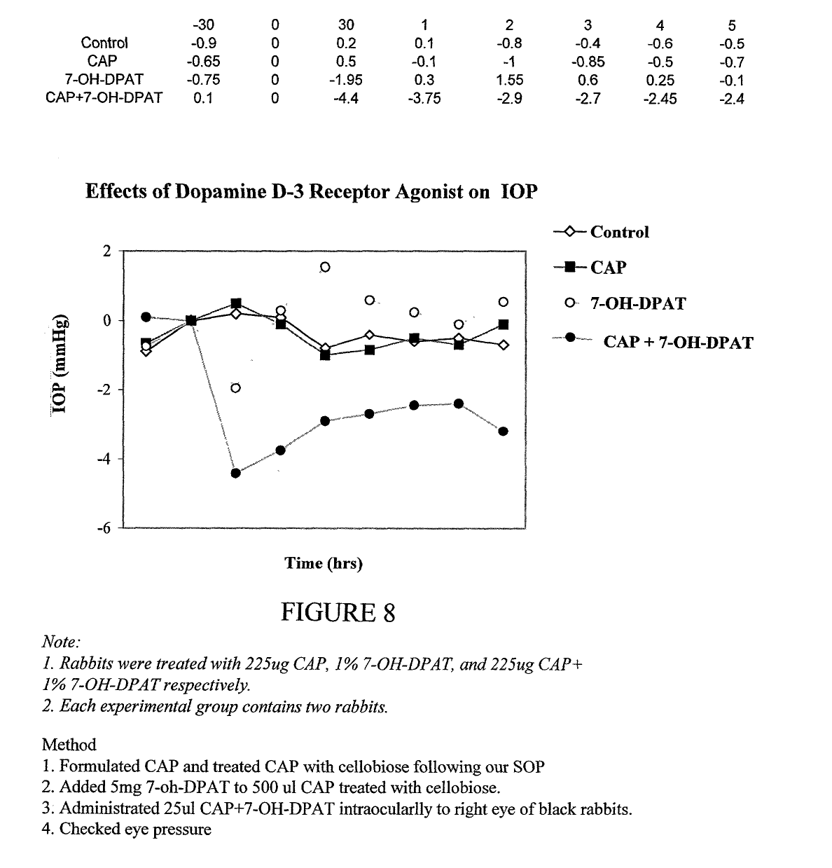

FIG. 8 shows the results of an experiment conducted to determine the effects on intraocular pressure resulting from intraocular delivery of dopamine combined with CAP.

### DETAILED DESCRIPTION OF SPECIFIC EMBODIMENTS OF THE INVENTION

The present invention relates to novel calcium phosphate core particles for mucosal delivery, to methods of making them, and to methods of using the core particles as vaccine adjuvants, as cores or carriers for pharmacologically active native and recombinant agents, and as controlled release matrices for pharmacologically active native and recombinant agents. The present invention also relates to the novel calcium phosphate core particles having a pharmacologically active agent at least partially coated on the surface of the core particles (“outside formulation”), or dispersed or impregnated within the core particles (“inside formulation”) or both (“outside/inside formulation”), to methods of making them, and to methods of using them.

Non-limiting examples of a pharmacologically active agents within the scope of this invention to be at least partially coated on the surface of the core particle or impregnated therein include vaccines, antigenic material, natural immunoenhancing factor(s), therapeutic monoclonal antibodies, oligonucleotide material encoding immunogenic polypeptides, therapeutic proteins or peptides, such as insulin, or other components capable of having a therapeutic effect when administered to a mucosal surface, which are discussed further below.

The embodiment of the invention directed to a CAP/antigen formulation can be used in active vaccination of humans or other mammals. As used herein, the term “antigenic material” or “antigen” means an immunogenic, native or recombinant antigen product obtained from a bacteria, virus, or fungus, and containing one or more antigenic determinants. Examples of antigenic material as this term is used herein include one or more portions of the protein coat, protein core, or functional proteins and peptides of a virus, such as Epstein-Barr virus (EBV), human immunodeficiency virus (HIV), human papilloma virus (HPV), herpes simplex virus (HSV), pox virus, influenza, or other virii, or immunogenic proteins obtained from bacteria, such as tuberculosis (TB), staphylococcal, streptococcal, clostridium, helicobacter, pseudomonas, or coliform bacterial antigens, or fungi, such as candida and other saccharomyces. The binding activity of calcium phosphate core particles allows a high loading capacity for these different types of proteins.

The invention is also useful to provide a vaccine against Pseudomonas aeruqinosa, Hemophilus influenzae, Helicobacter pylori, Vibrio cholerae, Bordetella pertussis, Corynebacterium diphtheriae, Escherichia coli, Salmonella and typhimurium, Clostridium perfringens and other enteric clostridiae, Shigella dysenteriae, Shiqella flexnerii Neisseria gonorrheae, Trichomonas, Entameba histolytica, Giardia lamblia, respiratory syncytial virus, rotavirus, reovirus, Human Immunodeficiency Virus, Human T-Cell Lymphotrophic Virus, Types I and II, polio virus, Rhinovirus, AIDS, pathogens such as Pneumocystis, and yeast such as monilia. In each case, an appropriate known antigen, e.g. whole pathogen or specific externally presented antigens such as the viral coat protein, or bacterial cell-surface proteins, pilus protein, lipopolysaccharides, viral capsid or envelope protein, protozoal plasma membrane surface component, spermatozoal surface proteins, or respiratory allergens are used. The invention may also be used to deliver recombinant DNA, that is either naked or contained in plasmid vectos, or to deliver RNA coding for microbial epitopes.

The invention is also useful to protect against allergens that contact the respiratory or ocular mucosal surfaces. Suitable ophthalmic agents which can be included in the compositions of the present invention and administered via the method of the present invention include, but are not limited to glaucoma agents, such as betaxolol, pilocarpine and carbonic anhydrase inhibitors; dopaminergic agonists; post-surgical antihypertensive agents, such as para-amino clonidine (apraclonidine); anti-infectives, such as ciprofloxacin; antimicrobials, such as cephalosporins and quinolones; non-steroidal and steroidal anti-inflammatories, such as suprofen, ketorolac and tetrahydrocortisol; prostaglandins; proteins; growth factors, such as EGF; immunosuppressant agents, and anti-allergics.

Exemplary pharmacologically active agents for delivery using the particles of the present invention may also include insulin, calcitonins (for example, porcine, human, salmon, chicken or eel) and synthetic modifications thereof, enkephalins, luteinizing hormone releasing hormone (LHRH) and analogues (such as Nafarelin, Buserelin, and Zoladex), growth hormone releasing hormone (GHRH), nifedipin, thymic humoral factor (THF), calcitonin gene related peptide (CGRP), atrial natriuretic peptide, antibiotics, metoclopramide, ergotamine, Pizotizin, nasal vaccines, (such as AIDS vaccines, influenza, pertussis, measles, rhinovirus Type 13 and respiratory syncitial virus), pentamidine and cholecystykinin (CCK).

Other exemplary compounds that can be delivered with the present invention include antibiotics, antimicrobial agents, and therapeutic monoclonal antibodies, such as tetracycline hydrochloride, leucomycin, penicillin, penicillin derivatives, erythromycin, sulphathiazole and nitrofurazone; local anaesthetics such as benzocaine; vasoconstrictors such as phenylephrine hydrochloride, tetrahydrozoline hydrochloride, naphazoline nitrate, oxymetazoline hydrochloride and tramazoline hydrochloride; cardiotonics such as digitalis and digoxin; vasodilators such as nitro-glycerine and papaverine hydrochloride; antiseptics such as chlorhexidine hydrochloride, hexylresorcinol, dequaliniumchloride and ethacridine; enzymes such as lysozyme chloride, dextranase; bone metabolism controlling agents such as vitamin D, and active vitamin D₃ ; sex hormones; hypotensives; sedatives; anti-tumor agents; steroidal anti-inflammatory agents such as hydro-cortisone, prednisone, fluticasone, prednisolone, triamcinolone, triamcinolone acetonide, dexamethasone, betamethasone, beclomethasone, and beclomethasone dipropionate; non-steroidal anti-inflammatory agents such as acetaminophen, aspirin, aminopyrine, phenylbutazone, mefanamic acid, ibuprofen, diclofenac sodium, indomethacin, colchicine, and probenocid; enzymatic anti-inflammatory agents such as chymotrypsin and bromelain seratiopeptidase; anti-histaminic agents such as diphenhydramine hydrochloride, chloropheniramine maleate and clemastine; anti-allergic agents and antitussive-expectorant antiasthmatic agents such as sodium chromoglycate, codeine phosphate, and isoproterenol hydrochloride; analgesics; and anti-migraine compounds.

The particles of the present invention can also be coated (with or without an intermediate coating of a surface modifying agent) or impregnated with natural immunoenhancing factors. These are typically proteins or peptides that function as natural adjuvants, stimulating the response of the immune system to antigenic challenge by a vaccine antigen. Suitable natural immunoenhancing factors include interleukins, including those already recognized to have immunoenhancing activity, such as interleukin-2 and interleukin-12, and those discovered in the future to have such activity.

Another embodiment of the present invention relates to calcium phosphate core particles modified to function as oligonucleotide vaccines, having DNA or RNA encoding immunogenic polypeptides at least partially coated on the surface of the core particles or at least partially impregnated therein. Exemplary oligonucleotide vaccines include those encoding immunogenic epitopes for influenza, malaria, colon cancer cells, hepatitis B, human immunodeficiency virus (HIV), simian immunodeficiency virus (SIV), cutaneous T cell lymphoma, herpes simplex, tick born encephalitis, rabies, rotavirus, tuberculosis, Epstein-Barr virus, human papilloma virus, and hepatomavirus. When administered to a patient, the core particle biodegrades and the DNA or RNA is taken up and expressed by the cells and translated to produce one or more immunogenic polypeptides that are recognized by the immune system.

Additionally, the compositions may include pharmacologically active antisense nucleotides, ribozymes, external guide sequences for RNAse P, or genes. The admixture of, or incorporation of the nucleic acid onto the surface, or into the core of the CAP improves the delivery of the nucleic acid to the site where it is needed. For example, the composition may be administered to and transported across mucosal surfaces for local and/or systemic delivery. The nucleic acid can be complexed with CAP, for example, by a covalent or ionic linkage.

In addition to the CAP and an antigen, ophthalmic agent, polynucleotide material or other pharmacologically active agent, compositions of the present invention may include other components. For example, pharmaceutically acceptable buffers, preservatives, nonionic surfactants, solubilizing agents, stabilizing agents, emollients, lubricants and/or tonicity agents may be included. The compositions of the present invention may be delivered via a spray, an aerosol, an ointment, an eye drop, a gel, a suspension, a capsule, a suppository, or an impregnated tampon, and so on. Those skilled in the art will understand how to formulate such vehicles by known techniques.

The core particles of the present invention may optionally have at least a partial coating of a surface modifying agent, which may help adhere the above-mentioned pharmacologically active agent to the core particle, or may have a surface modifying agent impregnating the particle, or both. A further aspect of the invention provides a method of treating a human or other mammal by administering a formulation as described above to a mucosal surface of that human or other mammal, for example the vagina, eye or nose.

I. Core Particles

The calcium phosphate core particles of the present invention have an average particle size between about 300 nm and about 4000 nm, more particularly, between about 300 nm and about 2000 nm. For the applications described herein, an average particle size of between about 300 nm and 1000 nm is sufficient and desirable. The core particles of the present invention have a morphology that is generally and substantially spherical in shape and a surface that is substantially smooth.

The term “substantially smooth” is used herein to mean essentially no surface features or irregularities having a size of 100 nm or larger. The core particles may be faceted or angular and still fall within this definition, as long as the facets do not contain many surface irregularities of the type described above. The term “substantially spherical” is used herein to refer to particles that are substantially round or oval in shape, and includes particles that are unfaceted and smooth, or that have very few facets, as well as particles that are polyhedral having several or numerous facets.

The following table provides a comparison between the calcium phosphate core particles of the present invention and calcium phosphate particles manufactured by Superfos Biosector a/s. The table shows that the calcium phosphate core particles of the present invention are small, smooth and ovoid, whereas Superfos Accurate CAP particles are large, jagged and crystalline.

| | BioSante | |
|---|---|---|
| | Pharmaceuticals, Inc. | Superfos Biosector a/s |
| | CAP | CAP |
| PH | 6.2-6.8 | 6.49 |
| Size | <1000 nm | >3000 nm |
| Morphology | Smooth ovoid shape | Jagged crystalline shape |

The calcium phosphate core particles of the present invention are typically prepared as a suspension in aqueous medium by reacting a soluble calcium salt with a soluble phosphate salt, and more particularly, by reacting calcium chloride with sodium phosphate under aseptic conditions. Initially, an aqueous solution of calcium chloride having a concentration between about 5 mM and about 300 mM is combined by mixing with an aqueous solution of a suitable distilled water-based solution of sodium citrate, having a concentration between about 5 mM and about 300 mM. The presence of sodium citrate contributes to the formation of an electrostatic layer around the core particle, which helps to stabilize the attractive and repulsive forces between the core particles, resulting in physically stable calcium phosphate core particles.

An aqueous solution of dibasic sodium phosphate having a concentration between about 5 mM and about 300 mM is then mixed with the calcium chloride/sodium citrate solution. Turbidity generally forms immediately, indicating the formation of calcium phosphate core particles. Mixing is generally continued for at least about 48 hours, or until a suitable core particle size has been obtained, as determined by sampling the suspension and measuring the core particle size using known methods. The core particles may be optionally stored and allowed to equilibrate for about seven days at room temperature to achieve stability in size and pH prior to further use.

In one embodiment, the calcium phosphate core particles of the present invention can be used without further modification as vaccine adjuvants. For instance, the core particles may be uncoated and can be administered in a dosage of about 1 μg to about 1000 μg per kilogram of total body weight in conjunction with killed, attenuated, or live vaccines, with decoy viruses, or with core particles at least partially coated with microbial antigenic material, such as those described above. The killed, live, or attenuated vaccines, decoy viruses, or antigen-coated core particles may be administered in the same solution as, or in a different solution from, that of the uncoated particles.

In another embodiment, the core particles of the present invention can also be at least partially coated or impregnated or both with a pharmacologically active agent, wherein the pharmacologically active agent is disposed on the surface of the core particle and optionally held in place by a surface modifying agent sufficient to bind the material to the core particle without denaturing the material. Non-limiting examples of the pharmacologically active agent discussed above.

In a further embodiment, the particles are complexed with surface modifying agents suitable for use in the present invention include substances that provide a threshold surface energy to the core particle sufficient to bind material to the surface of the core particle, without denaturing the material. Example of suitable surface modifying agents include those described in U.S. Pat. Nos. 5,460,830, 5,462,751, 5,460,831, and 5,219,577, the entire contents of each of which are incorporated herein by reference. Non-limiting examples of suitable surface modifying agents may include basic or modified sugars, such as cellobiose, or oligonucleotides, which are all described in U.S. Pat. No. 5,219,577. Suitable surface modifying agents also include carbohydrates, carbohydrate derivatives, and other macromolecules with carbohydrate-like components characterized by the abundance of —OH side groups, as described, for example, in U.S. Pat. No. 5,460,830. Polyethylene glycol (PEG) is a particularly suitable surface modifying agent.

The core particles may be at least partially coated by preparing a stock solution of a surface modifying agent, such as cellobiose or PEG (e.g., around 292 mM) and adding the stock solution to a suspension of calcium phosphate core particles at a ratio of about 1 mL of stock solution to about 20 mL of particle suspension. The mixture can be swirled and allowed to stand overnight to form at least partially coated core particles. The at least partially coated core particles are administrable alone or in conjunction with one or more of the materials described below. Generally, this procedure will result in substantially complete coating of the particles, although some partially coated or uncoated particles may be present.

II. Pharamcologically Active Agent Antigenic Material or Natural Immunoenhancing Factor

In one embodiment, the uncoated core particles or the core particles at least partially coated with surface modifying agent are then contacted with, impregnated with, or both, antigenic material or natural immunoenhancing factor, to produce particles having antigenic material or natural immunoenhancing factor at least partially coating the core particle. FIG. 1 is a schematic drawing of the particles of this embodiment, illustrating antigenic material or natural immunoenhancing factor (8) both coating the core particle (4) and incorporated within the core particle (4) (as will be discussed below). Antigen purified from viral coat or capsule proteins, or from cell surfaces of bacteria or fungi, can be obtained or purified using methods that are known in the art, or can be obtained commercially. For example, viral particles are obtained by infecting transforming host cell lines with the virus, and after a suitable incubation period, centrifuging the cell suspension and sonicating the resulting suspension at high power for several minutes to break open the cells, and again centrifuging the broken cell suspension. The supernatant containing virus can then be stored for further processing and protein purification using techniques familiar to those skilled in the art. Bacterial and fungal cell membrane antigens can be obtained by culturing and lysing the desired organisms and separating the desired antigenic protein fractions using techniques known in the art.

The antigen-coated particles of the invention are not produced by methods requiring the denaturing of the protein coating of a viral particle, removal of the core viral genetic material, and renaturing of the protein coating around a substitute core. Instead, the antigen-coated particles of the invention result from attachment of individual portions of protein coating to a calcium phosphate core. As a result, the particles of the invention are not believed to function as “decoy viruses” per se, as described in several of the patents cited above.

Instead, the particles of the invention can be more potent immunogenically than can a decoy virus, since only immunogenic portions of proteins need be attached to the particles. This increases the likelihood, for a given and a generally reduced (relative to alum) concentration of particles, that an antigenic epitope on the particle will elicit an immune response. In addition, the particles of the invention can be used to provide a broader spectrum of protection, since immunogenic material from several different pathogens can be attached to the surface of a single particle, or to the surfaces of different particles administered substantially simultaneously. These advantages are not obtained with the viral decoy particles described in the above-identified patents.

In addition to an antigen coating or in the alternative, the calcium phosphate core particles of the present invention can be prepared as controlled release particles for the sustained release of antigenic material or natural immunoenhancing factor over time, wherein the antigenic material or natural immunoenhancing factor (8) is incorporated into the structure of the core particle (4), shown in FIG. 1. This is done by mixing the aqueous calcium chloride solution with the antigenic material or natural immunoenhancing factor to be incorporated prior to combining and mixing with either the sodium citrate or dibasic sodium phosphate solutions, to co-crystallize the calcium phosphate core particles with the antigenic material or natural immunoenhancing factor. The antigenic material may consist of a native or recombinant immunogenic antigen product obtained from a bacteria, virus, or fungus, and containing one or more antigenic determinants, as described in detail above. The natural immunoenhancing factor may consist of proteins or peptides that function as a natural adjuvant, such as interleukins, particularly interleukin-2 and interleukin-12, also described in detail above.

In a more particular embodiment, the particles of this invention having an antigenic material complexed as an inside formulation, outside formulation, or inside/outside formulation may be administered to patients as a suppository, a gel, an eye drop, or any other delivery mechanism that allows contact of the formulation with a mucosal surface of the patient.

III. Pharamcologically Active Agent Polynucleotide Material

If polynucleotide material is coated onto and/or incorporated within the core particle, the particles function as an immediate- or controlledrelease matrix (or combination thereof) for the DNA or RNA. The DNA or RNA that is at least partially coated onto or incorporated within the core particles may be selected from a wide variety of DNA or RNA sequences that encode epitopes of one or more immunogenic polypeptides, and thus can be used as the active ingredient in a DNA or RNA vaccine. Antisense fragments may also be used. Exemplary polynucleotides include those encoding immunogenic epitopes for influenza, malaria, colon cancer cells, hepatitis B, human immunodeficiency virus (HIV), simian immunodeficiency virus (SIV), cutaneous T cell lymphoma, herpes simplex, tick born encephalitis, rabies, rotavirus, tuberculosis, Epstein-Barr virus, human papilloma virus, and hepatomavirus.

The polynucleotide can be naked or inserted into a plasmid vector. Suitable plasmids are known to those skilled in the art, and typically include pcDNA3 (Invitrogen), pCI (Promega) and pBR231. It may be desirable that the plasmid or naked DNA express cytomegalovirus (CMV) intermediate-early promoter, or bovine growth hormone polyadenylation sequence. A large number of expression vectors can be constructed by incorporating a cDNA sequence encoding an immunogenic polypeptide into a plasmid vector. The DNA or RNA segments may be prepared, inserted into vectors, and the vectors cloned according to known procedures, such as the procedures described in Maniatis, et al., Molecular Cloning, Cold Spring Harbor Laboratory Press, New York, 1.0-19.0 (1989). Gene segments are also available commercially from a number of different suppliers, and inserted into commercially available plasmids. When the sequence of a candidate protein is known, a coding sequence of the polynucleotide can typically be inferred and the corresponding gene segment prepared and isolated.

The polynucleotide sequence may be fused with other sequences in the vector, such as human tissue plasminogen activator leader peptide. The vectors can also include bacterial DNA or naked DNA surrounding the gene for the pathogenic antigen as a foreign sequence motif, increasing the immune response to that gene. See Y. Sato et al., Science 273:352-354 (1996); G. J. Weiner et al., PNAS 94 (20): 10833-7 (1997). Moreover, the plasmid may also include other genetic adjuvants, such as genes coding for cytokines, such as granulocyte-macrophage colony-stimulating factor (GM-CSF) or interleukins, to further multiply the immune response.

To form core particles having at least a partial coating of polynucleotide material, the at least partially coated core particles described above are contacted with polynucleotide material, i.e., DNA or RNA coding for one or more antigens expressed by organisms to be vaccinated against. When the core particles are coated, the DNA or RNA material is attached to the surface of the coating as described in U.S. Pat. No. 5,460,831. FIG. 2A shows a schematic drawing of the particles of this embodiment, with material (6), such as polynucleotide material coating the core particle (4).

In addition to a polynucleotide coating or in the alternative, polynucleotide material may be incorporated into the structure of the core particle. For example, the DNA or RNA coding for an epitope expressed on a viral protein coat or capsule can be mixed with a solution of calcium chloride, which can then be mixed with, e.g., a buffer, such as a sodium citrate solution, and a solution of dibasic sodium phosphate. The resulting particles will have the DNA or RNA dispersed or impregnated therein. A vector containing the DNA or RNA may also be added with one or more of the reactants forming the core particle, as described above. For example, a plasmid or other vector containing immunogen-encoding DNA or RNA or naked DNA can be mixed with the calcium chloride solution, so that the calcium phosphate biodegradable matrix forms around the plasmid or naked DNA, which becomes embedded in and/or on the core particle.

The impregnated or coated core particle fragments can be separated from the production mixture and stored for further use. Storage can be by any conventional methods for storing gene segments or antisense fragments. For example, the core particles may be lyophilized, spray-dried, or stored as a suspension in a compatible solution.

A typical polynucleotide vaccine produced according to the present invention contains about 0.5 to 500 micrograms of DNA or RNA material. When administered, the core particles are combined with a pharmaceutically acceptable carrier solution or other excipient. The dose will vary with the route of administration, the frequency of treatment, and other patient characteristics. Typical vaccination dosages include from about 0.1 mL to 2 mL of a vaccine containing about 0.5 to 500 micrograms of DNA or RNA material. Because the core particle supporting the DNA or RNA is biodegradable calcium phosphate, DNA or RNA that may impregnated therein is slowly released over time as the particles dissolve under physiological conditions. DNA or RNA released from the dissolving material is taken up and expressed by cells, and translated to produce one or more immunogenic polypeptides that are recognized by the humoral and cell-mediated immune system in the same manner as if the antigen had been vaccinated conventionally, but without the risks associated with the administration of live attenuated or killed virus. Moreover, the presence of calcium phosphate particles that have not completely dissolved serves an adjuvanting function for the DNA or RNA vaccine by enhancing the efficacy of the immunogenic protein or proteins expressed by the cells taking up the DNA or RNA.

IV. Pharamcologically Active Agent Therapeutic Protein or Peptide

In still a further embodiment, the at least partially coated core particles described above support a therapeutically effective protein or peptide. In addition, or in the alternative, the calcium phosphate core particles of the present invention can be prepared as controlled release particles for the sustained release of the therapeutic protein or peptide over time, wherein the therapeutic protein or peptide is incorporated into the structure of the core particle.

The core particles that are at least partially coated and/or impregnated with a therapeutic protein or peptide may function as an inhalable aerosol, as an ocular eye drop or ointment, as a vaginal suppository, or any of the mucosal delivery mechanisms described above. This protein or peptide may be any therapeutically effective protein or peptide, including but not limited to those listed above. Core particles coated or impregnated with a material (6), such as a therapeutic protein or peptide are shown in FIGS. 2 and 3.

Coating of the core particles with a therapeutic protein or peptide is preferably carried out by suspending the core particles in a solution containing a dispersed surface modifying agent, generally a solution of double distilled water containing from about 0.1 to about 30 wt % of the surface modifying agent. The cores are maintained in the surface modifying agent solution for a suitable period of time, generally about one hour, and may be agitated, e.g., by rocking or sonication. The at least partially coated core particles can be separated from the suspension, including from any unbound surface modifying agent, by centrifugation. The at least partially coated core particles can then be resuspended in a solution containing the protein or peptide to be adhered to the at least partially coated core particle. Optionally, a second layer of surface modifying agent may also be applied to the protein or peptide adhered to the particle.

In another embodiment, a protein or peptide may be attached to an unmodified particle surface, although particles at least partially coated with a surface modifying agent have greater loading capacities. For example, insulin loading capacities of at least partially coated particles have been found to be about 3 to 4-fold higher than insulin loading capacities of unmodified particle surfaces. Additionally, an increase in particle size may result in a greater loading capacity. For instance, an increase of 150 nm in particle size (relative to a starting size of 450 nm to 600 nm) results in about a 3-fold increase in insulin loading capacity in particles that are at least partially coated with a surface modifying agent.

Another embodiment that facilitates higher loading capacities is schematically illustrated in FIG. 2C, which shows a core particle having a surface modifying agent (2), such as polyethylene glycol, impregnated therein. The particles may be prepared by adding a surface modifying agent (2) to one or more of the aqueous solutions forming the core particle (4). The core particles may optionally be stored at room temperature. To obtain at least partially coated particles, the particles are subsequently contacted with a therapeutic protein or peptide, such as insulin, and more particularly human insulin, to provide at least a partial coating on the particle as described above.

A further embodiment facilitating higher loading capacities is illustrated in FIG. 3, which shows a core particle (4) having both a surface modifying agent (2), such as polyethylene glycol, and a material (6), such as therapeutic protein or peptide, more particularly insulin, and even more particularly human insulin, impregnated therein. One way in which particles of this embodiment may be prepared is by combining human insulin and/or other desired protein or peptide and a surface modifying agent together to form a solution. This solution is then combined with one or more of the aqueous solutions forming the particle as described above. The resulting particles incorporate calcium phosphate, surface modifying agent, and insulin within the core particle. Particles prepared according to this and any other embodiments described herein may be combined with one or more particles prepared according to any other embodiment described herein.

In a more particular embodiment, the composition of the present invention comprising a calcium phosphate core at least partially coated with polyethylene glycol and human insulin may be administered to diabetic patients as an aerosol of the dried particles, or as an aerosol of a solution of the particles in a carrier liquid, such as water. The particular insulin dose delivered may be lower or corresponds to that given intravenously and by other methods, and the dose of particulate insulin given is determined based on the blood glucose levels and supplied dosages of particles in the rat model described herein. Without wishing to be bound to the following dosage ranges, average daily doses of about 0.5 to about 2.0 mg are believed to be appropriate to generate a therapeutic effect in humans.

Incorporating a therapeutic protein or peptide into the particle may be accomplished by mixing an aqueous calcium chloride solution with the therapeutic protein or peptide to be incorporated prior to combining and mixing with either the sodium citrate or dibasic sodium phosphate solutions, to co-crystallize the calcium phosphate core particles with the therapeutic protein or peptide.

The particles, vaccines, and pharmaceutical compositions of this invention may be suitably administered to any patient in need thereof, namely to any species of animal that suffers or can suffer from the disease conditions described herein, more particularly mammals, and even more particularly humans.

Generally, our results demonstrate that mice immunized with CAP-based formulations of HSV-2 glycoprotein exhibited significantly increased survival rates and less severe clinical infection compared to controls, following challenge with a potentially lethal dose of live virus. These findings demonstrate that CAP delivered as a mucosal adjuvant confers protective antiviral immunity.

The various embodiments of the invention can be more clearly understood by reference to the following nonlimiting examples.

### EXAMPLE 1

A 12.5 mM solution of CaCl₂ is prepared by mixing 1.8378 g of CaCl₂ into 800 mL of sterile GDP water under aseptic conditions until completely dissolved, and the solution diluted to 1 L and filtered. A 15.625 mM solution of sodium citrate was prepared by dissolving 0.919 g of sodium citrate into 200 mL of sterile GDP water with mixing using aseptic techniques and filtered. A 12.5 mM solution of dibasic sodium phosphate was prepared by dissolving 1.775 g sodium phosphate into 1 L of sterile GDP water with mixing using aseptic techniques and filtered. All solutions were stored at room temperature.

The calcium chloride solution was combined with the sodium citrate solution and thoroughly mixed. Subsequently, the sodium phosphate solution was added with mixing. Turbidity appeared immediately as particles began to form. The suspension was allowed to mix for several minutes and was sampled for endotoxin testing using aseptic technique. Mixing was continued for about 48 hours under a laminar flow hood. Following mixing, the particles were sonicated on a high power setting for about 30 minutes at room temperature, tested for endotoxin concentration and pH and characterized as to particle size with a Coulter N4Plus Submicron Particle Sizer. Photomicrographs of particles prepared in this way are shown in FIGS. 1A and 1B. Following preparation the particles were allowed to equilibrate for approximately seven days before use.

### EXAMPLE 2

An HSV-2 protein solution and an Epstein-Barr virus (EBV) protein solution were purified from ATCC VR-540 (infected tissue culture fluid and cell lysate). The viral suspension was contacted with a lysis buffer (1% IGEPAL CA-630 for HSV-2 and 1% Triton x 100 for EBV, 10 mM NaCl, 10 mM Tris-HCL, and 1.5 mM MgCl₂), vortexed for 1 minute, incubated on ice for 30 minutes, and centrifuged at 1400 rpm for 2 hours at 4° C. The resulting supernatant was then contacted with a second lysis buffer (1 mM PMSF, 1% IGEPAL CA-630 for HSV-2 and 1% Triton x 100 for EBV, 100 mM NaCl, 100 mM Tris-HCL, and 3 mM MgCl₂), incubated on ice for 30 minutes, and centrifuged at 1400 rpm for 2 hours. The supernatant was then dialyzed against 2L of 0.9% saline overnight, lyophilized and resuspended in 1 mL PBS.

### EXAMPLE 3

The HSV-2 protein of Example 2 was added to 75 ml or 12.5 mM calcium chloride, followed by the addition of 75 ml of 12.5 mM dibasic sodium phosphate and 15 ml of 15.6 mM sodium citrate similar to the particle formation methods described in Example 1. The solution was stirred until the final average particle size was less than 1,200 nm, as determined with a Coulter N4Plus Submicron Particle Sizer. The particle mixture containing entrapped HSV-2 protein was treated with cellobiose overnight and mixed again with 600 μg HSV-2 protein for 1 hour at 4° C. After washing off unbounded proteins with PBS, the HSV2-2+CAP vaccine formulation was ready for use.

### EXAMPLE 4

The efficacy of the particles prepared as described by Example 3 was tested as follows: Female BALB/c mice 6 to 8 weeks old, and 25 g were obtained from Charles River Laboratories. The mice were maintained in standard housing with a normal diet of Purina rodent chow 5001.

Six groups of 5 female BALB/c mice were inoculated intravaginally (Ivag) or intranasally (IN) with HSV-2+CAP (20 μg protein plus 51 μg CAP per dose/mouse), HSV-2 alone (20 μg per dose/mouse), or CAP alone (51 μg per dose/mouse) in total volume of 50 μl. The mice received two inoculations at one-week intervals, on days 0 and 7. Samples were collected at 14, 21, and 45 days after the first immunization. Blood was obtained from the orbital sinus, and the sera was stored at −20 ° C. Mucosal samples were collected by vaginal and oral lavage with 100 μl of PBS. The sediments were removed by centrifugation, and samples were pooled and stored at −20° C.

### EXAMPLE 5

Serum and mucosal samples were analyzed by standard enzyme-linked immunosorbant assay (ELISA) for anti-HSV-2 IgG, IgG2a and IgA antibodies. Microtiter plates (Corning; Cambridge, Mass.) were coated with HSV-2 (6 μg/ml) overnight at 4° C. and blocked with PBS containing 0.05% Tween 20 and 0.1% normal goat serum. Diluted sera were incubated with antigens, washed, then incubated with horse radish peroxidase-conjugated goat anti-mouse second antibody-IgG (Chemicon; Temecula Calif.) and IgA, (Fisher Scientific Co; Pittsburgh, Pa.). Optical densities were read at 490 nM using a Benchmark Microplate Reader (BioRad; Hercules, Calif.)

### EXAMPLE 6

Mice were injected subcutaneously with DepoProvera (Upjohn; Kalamazoo, Ml) at a concentration of 2 mg/mouse in 50 μl of distilled water on the 45^{th} day following primary immunization. Five days later, on the 50^{th} day after primary immunization, the mice were challenged intravaginally with 10⁶ PFU of HSV-2. Mice were examined daily for genital pathology, and the clinical scoring was performed by an investigator blinded to the animal's immunization status. Clinical pathology was scored on a 5-point scale: 0, no apparent infection; 1, slight redness of external vagina; 2, severe redness and swelling of external vagina; 3, genital ulceration with severe redness, swelling, hair loss of genital and surrounding tissue; 4, severe ulceration of genital and surrounding tissue and paralysis; 5, death. The results are shown in FIG. 6.

### EXAMPLE 7

Vero cells were propagated in culture plates. Pooled mice sera, after complement inactivation, were incubated with HSV-2, then assessed for the presence of HSV-2 specific neutralizing antibodies by plaque assay. The titer is the reciprocal of the serum dilution required to inhibit the cytolysis of a confluent monolayer of Vero cells by 50%.

The above examples generally describe methods used to evaluate three vaccine formulations for antibody and protective responses in mice: HSV-2 alone, CAP alone, and HSV-2+CAP. As indicated in FIG. 4a, at 21 days after primary immunization, intranasal delivery of HSV-2+CAP resulted in higher anti-HSV-2 vaginal mucosal IgG and IgA titers as compared to HSV-2 or CAP alone, although these differences were not significant. Similarly, intravaginal delivery of HSV-2+CAP resulted in higher anti-HSV-2 vaginal mucosal IgG and IgA titers compared to HSV-2 alone or CAP alone (FIG. 4b).

Serological IgG titers performed sequentially on days 14, 21 and 45 show an increasing systemic response in the mice intranasally immunized with HSV-2+CAP, when compared to CAP or HSV-2 alone, although the rise is not statistically significant (FIG. 5a). Anti-HSV-2 serum IgG2a titers were similar across the three groups (data not shown). However, the animals intravaginally immunized with HSV-2+CAP showed no difference in serum IgG HSV-2 antibody titers over time compared to the control groups (FIG. 5b). Titers for anti-HSV-2 serum IgG2a were similar across the three groups (data not shown).

The neutralization assay, which tested for HSV-2 specific neutralizing antibodies in the sera of immunized mice, was performed at day 21, or 2 weeks following secondary immunization. Neutralization antibodies were found in both the intranasal and intravaginal HSV-2+CAP immunized mice at titers 1:40 and 1:80, respectively, correlating with the increased IgA response also seen in the body fluids of these groups. Notably, neutralizing antibodies were absent in the CAP, intranasal HSV-2, and intravaginal HSV-2 inoculated mice. Day 50 after the primary vaccine, each mouse was administered a live viral challenge. Resistance to HSV-2 infection was evaluated by monitoring clinical pathology.

The intravaginal vaccination with HSV-2+CAP resulted in significantly (p<0.05) lower pathological severity on days 8, and 10 as compared to HSV-2 or CAP alone (FIG. 6b). On day 6, the reduced clinical severity in those HSV-2+CAP intravaginally immunized mice achieved statistical significance only when compared to the HSV-2. None of the mice intravaginally inoculated with HSV-2+CAP died from HSV-2 infection, whereas all of the mice intravaginally vaccinated with HSV-2 alone and CAP alone developed severe disease and died by Day 8 or 10. Similarly, those mice intranasally vaccination with HSV-2+CAP showed reduced clinically severity when compared to mice immunized with HSV-2 alone or CAP alone at Days 8 and 10 (FIG. 6a). However, there was no significant difference among the groups. As expected, the mice receiving CAP or HSV-2 alone were not protected against viral infection. Hence, the presence of CAP as an adjuvant in the HSV-2+CAP vaccine generated vaccine efficiency compared to HSV-2 or CAP alone, conferring statistically significant protection against a lethal dose of HSV-2 in the intravaginally immunized group.

The current inventors have previously reported that CAP delivered intraperitoneally with HSV-2 and Epstein-Barr virus (EBV) proteins induced high titers of IgG2a antibody, neutralizing antibody, and facilitated a high percentage of protection against viral infection in a murine model. See He Q, Mitchell AR, Johnson SL, Morcol T, Bell SJ, Calcium phosphate nanoparticles adjuvant, Clinical and Diagnostic Laboratory Immunology, November. 2000: 899-903; U.S. patent application Ser. No 09/496,771. In the current invention, however, the present inventors evaluated the immunogenicity and efficacy of a mucosal vaccine utilizing CAP as a vaccine adjuvant with HSV-2 protein. The results indicated that the HSV-2+CAP formulation induced higher levels of genital anti-HSV-2 IgA titers compared to controls. This is an important finding since IgA antibody response is thought to play a major role in preventing the attachment of pathogens to the epithelial surface and conferring protection against subsequent viral infection. Intranasal and intravaginal immunization routes were effective for the HSV-2+CAP formulation.

The data suggest that the mice intravaginally immunized with HSV-2+CAP had greater resistance against systemic HSV-2 infection, despite the inability to document a rise in the serum IgG. Nonetheless, vaginal secretions of HSV-2+CAP immunized rodents were found to have not only elevated IgA HSV-2 specific antibodies, but also IgG specific antibodies. Additionally, neutralizing antibodies were found in the HSV-2+CAP inoculated animals. The findings indicate that CAP appears to enhance the immunogenicity of HSV-2 alone, in inducing mucosal and systemic immunity.

The particles of the present invention may be delivered via any mucosal route, although some routes have been shown to be more effective than others. For example, intranasal administration paradoxically conferred more systemic immunity in inducing higher IgG titers, while the intravaginal route conferred greater IgA levels. Both routes, however, showed neutralizing antibodies, and disease resistance to systemic HSV-2 infection, but the intravaginally inoculated mice had superior survival when compared to their intranasal counterparts.

### EXAMPLE 8

The inventors also measured antibody and protective responses in mice immunized with influenza M1 protein with or without CAP. Mice treated with CAP+M1 induced high level IgG and IgG2a antibody in serum sample, as well as high IgA in mouth wash sample (FIG. 7a). Morbidity was evaluated by measuring body weight every other day (FIG. 7b). At day 10 and 12 the difference of body weight between CAP+M1 vaccinated mice and the control groups was statistically significant. Animals that received intranasal immunization of M1+CAP were 100% protected and suffered less disease than control mice (FIG. 7c). The CAP-alone group produced 40% lethality.

The Influenza M1+CAP formulation also induced higher levels of IgG2a and IgA. It has not been previously reported that M1+CAP delivered mucosally would induce antibody responses.

### EXAMPLE 9

A preliminary investigation studying a dopamine D-3 receptor agonist combined with CAP for intraoccular delivery and its effects on intraocular pressure was also conducted. The results of the experiment are shown in FIG. 8. Generally, the results indicate that rabbits treated with CAP combined with dopamine (either inside, outside, or inside/outside formulations) exhibit lower introacular pressure over time as compared to rabbits that either remained untreated or that were treated with CAP alone or with a dopamine D-3 receptor agonist alone.

The particles that were used in this specific experiment were prepared as discussed below, but it should be understood that any of the methods described herein could be used to prepare effective particles. CAP particles were prepared according to the methods described in Example 1. Cellobiose was also added during the formation. Once the particles formed, 5 mg of 7-OH-DPAT was added to 500 microliters of the CAP/cellobiose particles.

The test the efficacy of the particles, an effective amount, in this case, 25 microliters was delivered intraocularly to an eyes of rabbits and the eye pressure was checked. Rabbits receiving the CAP+7-OH-DPAT particles exhibited a clear decrease in eye pressure.

The particular embodiments of the invention having been described above are not limiting of the present invention, and those of skill in the art can readily determine that additional embodiments and features of the invention are within the scope of the appended claims and equivalents thereto.

What is claimed is:

1. A particle comprising calcium phosphate having a pharmacologically active agent at least partially coating the particle or impregnating the particle or both, wherein the particle has a diameter between about 300 nm and about 4000 nm and is adapted to be delivered to a mucosal surface.

2. The particle of claim 1, wherein the pharmacologically active agent is selected from the group consisting of vaccines, antigenic materials, natural immunoenhancing factors, oligonucleotide material encoding immunogenic polypeptides, therapeutic drugs, and combinations thereof.

3. The particle of claim 1, wherein the pharmacologically active agent is herpes simplex-2.

4. The particle of claim 1, further comprising a pharmaceutically acceptable excipient.

5. A method for inducing immunity in a patient, comprising delivering a particle of claim 1 to a mucosal surface of the patient in need thereof, wherein the pharmacologically active agent is an antigen.

6. The method of claim 5, wherein the mucosal surface is selected from the group consisting of a nasal surface, upper airway, lower airway, pulmonary surface, oral surface, ocular surface, vaginal surface, and rectal surface.

7. The method of claim 5, wherein the particle is complexed with a pharmaceutically acceptable excipient and delivered as a spray, an aerosol, an ointment, an eye drop, a gel, a suspension, a capsule, a suppository, an impregnated tampon, or combinations thereof.

8. A method for preparing particles suitable for eliciting a mucosal immune response, comprising:
(a) mixing an aqueous solution of calcium chloride with an aqueous solution of sodium citrate to form a mixture;
(b) adding an aqueous solution a sodium phosphate to the mixture to form a solution;
(c) stirring the solution until particles of the desired size and comprising calcium phosphate are obtained; and
(d) (i) contacting the particles with an antigenic material to form particles that are at least partially coated with the antigenic material, (ii) adding antigenic material with the aqueous solution of calcium chloride, the aqueous solution of sodium citrate, or the aqueous solution a sodium phosphate to form particles that have antigenic material at least partially impregnating the particle, or (iii) both (i) and (ii) to form particles that are at least partially coated and at least partially impregnated with antigenic material.

9. The method of claim 8, wherein the stirring comprising sonicating.
